(19) United States
(12) Patent Application Publication
(10) Pub. No.: US 2006/0280697 A1
(43) Pub. Date: Dec. 14, 2006

(54) Composition

(75) Inventor: Peter Freunscht, Casalpusterlengo (IT)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/450,489

(22) Filed: Jun. 9, 2006

(30) Foreign Application Priority Data
Jun. 10, 2005 (EP) EP05076360

(51) Int. Cl. A61K 8/49; A61K 8/97

(52) U.S. Cl. 424/49; 424/58

(57) ABSTRACT

An oral care composition, comprising: a therapeutically effective amount of a cooling compound having the structure of Formula [I]: 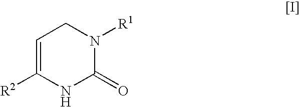
or a salt thereof to produce a cooling sensation, wherein R¹ and R² are independently selected from hydrogen or halogen atoms; hydroxy, cyano, nitro, mercapto, carbonyl, sulfone and carboxy groups; or optionally substituted alkyl, alkenyl, alkoxy, alkylthio, aryl, aryloxy, arylthio, amino, siloxy, ester and heterocyclic groups, characterised by an oil soluble agent for solubilising or emulsifying the cooling compound.

The present invention relates to an oral care composition comprising a flavour and a 1-R1-phenyl, 4-R2-phenyl substituted 1,2,3,6-tetrahydropyrimidine-2-one cold receptor agonist.

WO 2004/026840 (Unilever) discloses the use of a compound according to Formula [PA1]: 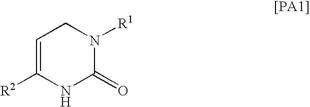
or a salt thereof to produce a cooling sensation, wherein R¹ and R² are independently selected from hydrogen or halogen atoms; hydroxy, cyano, nitro, mercapto, carbonyl, sulfone and carboxy groups; or optionally substituted alkyl, alkenyl, alkoxy, alkylthio, aryl, aryloxy, arylthio, amino, siloxy, ester and heterocyclic groups, with the proviso that when R¹ is 2-hydroxyphenyl, R² is other than 3-nitrophenyl. WO 2004/056332 (Unilever) discloses a composition comprising:
(a) from 0.005% to 0.5% by weight of a cooling compound;
(b) from 0.1% to 10% by weight of an emulsifiable substance;
(c) from 0.15% to 15% by weight of a surfactant;
(d) optionally up to 5% by weight, preferably from 0.05% to 5% by weight of a cosurfactant.

WO 2003/092697 (Cragmont Pharmaceuticals) discloses a therapeutic composition is provided that comprises a 1-R1-phenyl, 4-R2-phenyl substituted 1,2,3,6-tetrahydropyrimidine-2-one cold receptor agonist in a therapeutically effective amount. The composition preferably further comprises one or more pharmaceutically active drugs such as an anti-inflammatory glucocorticosteroid, a sympathomimetic amine decongestant, an antihistamine, a local anesthetic, menthol or a menthol analog, an immunosuppressant, and mixtures thereof. The cold receptor agonist may be represented by the general formula (PA2)-[R1-phenyl]-4-[R2-phenyl]-1,2,3,6-tetrahydropyrimidine-2-one wherein: R1 is -hydroxy, -chloro, -fluoro, -alkyl, -acetoxy, -trifluoromethyl; and R2 is -nitro, -chloro, -fluoro, -alkyl, -trifluoromethyl. Therapeutic compositions of the invention elicit long-lasting cooling or soothing, particularly when formulated for delivery to suppress the sensations of itch and pain, such as for delivery to inflamed skin, to the mucous membranes of the eyelids, of the anogenital areas and of the airways, and to the enteric mucosa.

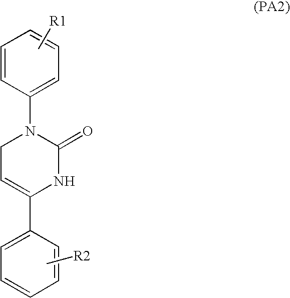

A known compound for producing a sensation of cold is menthol (2-isopropyl-5-methyl-cyclohexanol), which has been extensively applied as an additive in, for example, foodstuffs and oral hygiene products. It is used primarily because it elicits a sensation of coolness in the mouth, and because it has a pleasing mint flavour and odour. The cooling effect of menthol is due to the action of menthol on the nerve endings of the human body which detect hot and cold stimuli. In particular, menthol is believed to activate cold receptors on nerve endings. However, the use of menthol is limited by its strong minty smell and relative volatility.

It was found that icilin (also known as AG-3-5, chemical name 1-[2-hydroxyphenyl]-4-[2-nitrophenyl]-1,2,3,6-tetrahydropyrimidine-2-one) was capable of producing the same cooling effect as menthol. Icilin has a number of advantages over menthol, for example it is more potent, and has a lower acute toxicity, due to its lack of anaesthetic properties. Icilin was considered to be a particularly useful compound for pharmacological applications because it lacks the flavour and odour of menthol and is not readily absorbed through the skin. However, icilin has not been disclosed as a replacement for menthol for non-pharmaceutical applications.

According to a first aspect to the invention there is provided an oral care composition comprising a compound according to Formula [I]: 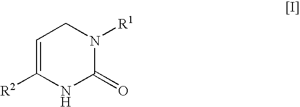
or a salt thereof to produce a cooling sensation, wherein R¹ and R² are independently selected from hydrogen or halogen atoms; hydroxy, cyano, nitro, mercapto, carbonyl, sulfone and carboxy groups; or optionally substituted alkyl, alkenyl, alkoxy, alkylthio, aryl, aryloxy, arylthio, amino, siloxy, ester and heterocyclic groups, characterised by an oil soluble agent for solubilising or emulsifying the cooling compound.

Preferably when R¹ is 2-hydroxyphenyl, R² is other than 3-nitrophenyl.

Preferred R¹ groups include optionally substituted alkyl or aryl groups. The alkyl group can be a linear group such as a C₁₋₁₀ aliphatic chain, or a cyclic group, such as a C₃₋₁₀ cyclic hydrocarbon. It is preferred that R¹ is an optionally substituted aryl or cyclic hydrocarbon group, such as a phenyl or cyclohexyl group.

The preferred R² groups include hydrogen atoms, or optionally substituted alkyl or aryl groups. Again, the R² group can be a linear, aliphatic chain, or a cyclic hydrocarbon, as for R¹. The preferred groups are optionally substituted aryl and cyclic hydrocarbon groups, with phenyl and cyclohexyl being particularly favoured.

Alternatively, it may be desirable to replace these cyclic groups with other groups, such as a hydrogen atom, a straight chain alkyl group (e.g. a C₁₋₁₀ alkyl group) or a branched chain alkyl group (e.g. a tertiary butyl group). A subset of particularly preferred compounds in the first aspect of the invention are those according to general formula [II]: 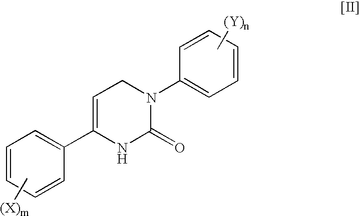
wherein the or each X and Y is independently a halogen atom or an alkyl, alkenyl, haloalkyl, alkoxy, hydroxy, thiol, carboxy, nitro, sulphonamide, sulphonato, sulphonyl, alkoxycarbonyl, carbonyl or amino group, and m and n are independently 0, 1, 2 or 3, preferably with the proviso that when n is 1, m is 1 and Y is a hydroxy group in the ortho position, X is other than a nitro group in the meta position. Where the substituent X or Y groups contain an alkyl portion (e.g. the alkyl portion of haloalkyl), this alkyl portion preferably contains from 1 to 6 carbon atoms. In the case of an alkenyl group, this preferably contains from 2 to 6 carbon atoms.

X can be selected from any of the groups listed above, with hydrogen and halogen atoms, and nitro, hydroxy, C₁₋₆ alkyl, C₁₋₆ haloalkyl and C₁₋₆ alkoxy groups being preferred. The number of X substituents can vary between 0 and 3, and the point of substitution of the phenyl ring may also be varied. It is preferred that there is a single substituent on the phenyl ring, i.e. where m is 1. In this case the substituent can be present in the ortho, meta or para position, relative to the point of attachment of the phenyl ring to the rest of the molecule containing the cyclic urea group. The optimal point of attachment will depend on a number of factors, such as the nature of the substituent and its electron-donating or electron-withdrawing effect. Particularly useful compounds include those where the substituent is present in the meta position.

Similarly, Y can be selected from any of the groups listed above, with hydrogen, hydroxy, C₁₋₆ alkyl, C₁₋₆ haloalkyl and C₁₋₆ alkoxy groups being preferred. The number of Y substituents can vary between 0 and 3, and the point of substitution of the phenyl ring may also be varied. It is preferred that there is a single Y substituent (i.e. where n is 1), which may be present in the ortho, meta or para position, relative to the point of attachment of the phenyl ring to the rest of the molecule containing the cyclic urea group. Particularly useful compounds include those where the single Y substituent is present in the ortho position. Thus, a particularly preferred compound in accordance with the first aspect to the invention is one according to general formula [III]: 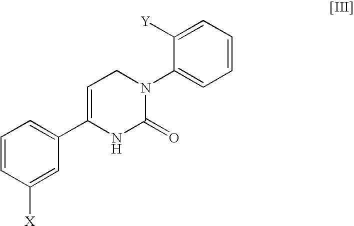
wherein X and Y are independently selected from a halogen atom or an alkyl, alkenyl, haloalkyl, alkoxy, hydroxy, thiol, carboxy, nitro or amino group.

Preferably, when Y is a hydroxy group, X is other than a nitro group. It is preferred that X is a hydrogen or halogen atom, or a nitro, hydroxy, C₁₋₆ alkyl or C₁₋₆ alkoxy group. Particularly preferred groups include halogen atoms, and methyl, ethyl, methoxy and ethoxy groups. The preferred halogen atom is chlorine.

A number of compounds have been synthesised, and show an ability to elicit a cooling sensation. Preferred compounds include 1-(2′-methoxyphenyl)-4-(3″-nitrophenyl)-1,2,3,6-tetrahydropyrimidine-2-one, 1-phenyl-4-(3″-nitrophenyl)-1,2,3,6-tetrahydropyrimidine-2-one, 1-(2′-methoxyphenyl)-4-(3″-chlorophenyl)-1,2,3,6-tetrahydropyrimidine-2-one, 1-phenyl-4-(3″-chlorophenyl)-1,2,3,6-tetrahydropyrimidine-2-one, 1-(2′-methylphenyl)-4-(3″-nitrophenyl)-1,2,3,6-tetrahydropyrimidine-2-one, 1-(2′-methoxyphenyl-4-(3″-methoxyphenyl)-1,2,3,6-tetrahydropyrimidine-2-one, 1-phenyl-4-(3″-methoxyphenyl)-1,2,3,6-tetrahydropyrimidine-2-one, 1-(2′-hydroxyphenyl)-4-(3″-methoxyphenyl)-1,2,3,6-tetrahydropyrimidine-2-one, 1-(2′-trifluoromethylphenyl)-4-(3″-nitrophenyl)-1,2,3,6-tetrahydropyrimidine-2-one, 1-phenyl-4-(3″-nitrophenyl)-1,2,3,6-tetrahydropyrimidine-2-one and 1-(2′-hydroxyphenyl)-4-phenyl-1,2,3,6-tetrahydropyrimidine-2-one. In particular, 1-(2′-hydroxyphenyl)-4-(3″-methoxyphenyl)-1,2,3,6-tetrahydropyrimidine-2-one, 1-(2′-trifluoromethylphenyl)-4-(3″-nitrophenyl)-1,2,3,6-tetrahydropyrimidine-2-one, 1-phenyl-4-(3″-nitrophenyl)-1,2,3,6-tetrahydropyrimidine-2-one and 1-(2′-hydroxyphenyl)-4-phenyl-1,2,3,6-tetrahydropyrimidine-2-one have shown good cooling effects.

The cooling compound is suspended, emulsified or solubilised in a composition by the oil soluble agent. The nature of additional substances, and the relative proportions of components of the composition will depend on a number of factors, such as the specific use for which the composition is employed. The compositions may be used in a variety of applications, such as those discussed above. Particularly preferred uses include personal hygiene products such as deodorant, shower gel and skin cream; oral hygiene products such as toothpastes and mouthwashes; and foodstuffs, such as beverages, ice-creams, confectionery and spreads.

Preferably, there is provided an oral care composition comprising a compound of formula [IV]: 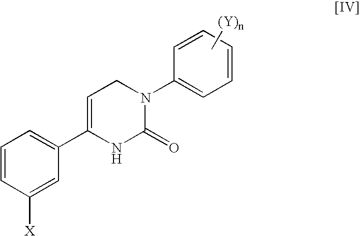
or a salt thereof, wherein X is a hydrogen or halogen atom, or a hydroxy, nitro, alkyl or alkoxy group; Y is hydrogen, hydroxy, haloalkyl, nitro or alkoxy; and n is 0, 1, 2 or 3, preferably with the proviso that when n is 1 and Y is hydroxy, X is alkyl or hydroxy. It is preferred that when n is 1 and Y is hydroxy, X is not a halogen atom. According to one aspect of this embodiment, it is preferred that when n is 1 and Y is hydroxy, X is alkyl or hydroxy. Particularly preferred in this aspect is where Y is hydroxy.

In other embodiments it is preferred that Y is haloalkyl, particularly when X is nitro. A particularly preferred haloalkyl is halomethyl, most preferably trifluoromethyl.

The number of Y substituents may vary between 0 and 3, and the substituents can be present in any position. However, it is preferred that there is a single Y substituent, i.e. where n is 1. This substituent may be present in the ortho, meta or para position, relative to the point of attachment of the phenyl ring to the rest of the molecule. The optimal position of the substituent will depend on a number of factors, such as the nature of the substituent and its electron-donating or electron-withdrawing effect. The ortho position is preferred.

Thus, particularly preferred compounds are those according to the general formula [V]: 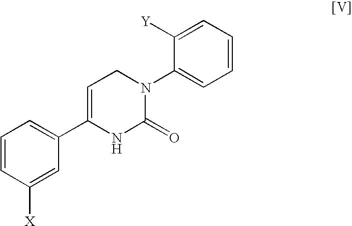
with Y being as defined above. It is preferred that Y is a hydroxy group or a methoxy group. X is preferably a hydrogen or halogen atom, or a hydroxy, C₁₋₆ alkyl or C₁₋₆ alkoxy group, providing that where Y is hydroxy, X is alkyl or hydroxy. Particularly preferred X groups include halogen atoms, and methyl, ethyl, methoxy and ethoxy groups. Chlorine atoms and methoxy groups are most preferred. Compounds according to this embodiment which have been shown to exhibit a cooling effect include those listed above in relation to the first embodiment, particularly 1-(2′-hydroxyphenyl)-4-(3″-methoxyphenyl)-1,2,3,6-tetrahydropyrimidine-2-one, 1-(2′-trifluoromethylphenyl)-4-(3″-nitrophenyl)-1,2,3,6-tetrahydropyrimidine-2-one, 1-phenyl-4-(3″-nitrophenyl)-1,2,3,6-tetrahydropyrimidine-2-one and 1-(2′-hydroxyphenyl)-4-phenyl-1,2,3,6-tetrahydropyrimidine-2-one. Most preferred are 1-(2′-hydroxyphenyl)-4-(3″-methoxyphenyl)-1,2,3,6-tetrahydropyrimidine-2-one and 1-(2′-trifluoromethylphenyl)-4-(3″-nitrophenyl)-1,2,3,6-tetrahydropyrimidine-2-one.

Again, the compounds the invention may be used in a variety of applications, such as those discussed above. In particular they may be used in applications similar to those described above in relation to the first embodiment of the invention, for example in personal hygiene products such as deodorant, shower gel and skin cream; oral hygiene products such as mouthwash and toothpaste; and food products. The compounds may have particularly useful applications in foodstuffs such as beverages, spreads, confectionery and ice-cream.

The second aspect to the present invention provides novel compositions comprising compounds of the invention. The compositions can be used for a number of applications where a cooling sensation is desirable. As discussed above, such applications may include the fields of personal hygiene products (including lotions, shaving cream, post shaving preparations, shampoos, conditioners, facial cleansers, soaps, bath oils and foams, antiperspirants and deodorants); oral hygiene products (including toothpastes, mouthwashes, dental floss, chewing gum and breath fresheners); food products (including beverages, spreads, ice-creams and confectionery); and other applications where a cooling sensation may be desirable (including pharmaceutical products such as chewable pharmaceutical products or throat lozenges, tobacco products, insect repellents and cosmetics).

Particularly preferred are compositions for use as toothpastes, mouthwashes and food products such as confectionery, beverages, spreads and ice-cream. The specific nature of the composition (e.g. the nature of the additional components, the relative proportions of the components and the physical nature of the composition) will depend on the particular application.

Preferably the composition is a composition such as a toothpaste, mouthwash or food product composition, comprising a compound of formula [I]: 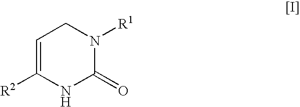
or a salt thereof to produce a cooling sensation, wherein R¹ and R² are independently selected from hydrogen or halogen atoms; hydroxy, cyano, nitro, mercapto, carbonyl, sulfone and carboxy groups; or optionally substituted alkyl, alkenyl, alkoxy, alkylthio, aryl, aryloxy, arylthio, amino, siloxy, ester and heterocyclic groups. In this aspect it is preferred that the compound of formula [I] is other than a compound where R¹ is 2-hydroxyphenyl and R² is 3-nitrophenyl.

The compound of formula [I] can be any compound resulting from a selection of R¹ and R² from the list given above. However, preferred compounds are those which have already been discussed in relation to the first embodiment of the invention above.

The oral care composition according to the invention, which may be any form of oral composition known in the art, e.g. a toothpaste, tooth powder, gel, foam, lozenge, etc may comprise further ingredients which are common in the art, such as:

antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. Preferred abrasives are chalk and silica, more preferably fine ground natural chalk.

Humectants such as glycerol, sorbitol, propyleneglycol, xylitol, lactitol etc.;

binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oil soluble agent is preferably a flavour component. Typical flavour components include those selected from oils and extracts of basil, camphor, caraway, cardamon, coriander, geranium, ginger, laurel, lavender, mace, menthol, nutmeg, pepper, rose, rosemary, thyme, ylang ylang, jasmin, vanilla, hyssop, lavandin, orris, carrot seed, davana, elemi and osmanthus; borneol and its derivatives; heliotropin, α-ionone, β-ionone, γ-ionone, δ-ionone and derivatives thereof; lactones and thymol, vanillin, ethyl vanillin, maltol and ethyl maltol. Preferably the suspending agent is present in the composition at from 0.001 to 2% by weight, more preferably from 0.1 to 1% by weight and most preferably from 0.25 to 0.75% by weight of the composition.

Preferably, the cooling agent is suspended, emulsified or solubilised in the oil soluble agent. More preferably, the oil soluble agent solubilises or emulsifies the cooling compound. Most preferably, it emulsifies the cooling agent.

In a third aspect the present invention presents a method for manufacturing an oral care composition comprising a cooling agent as described in the first aspect of the invention. The method is characterised by the mixing of the cooling agent in an oil soluble agent as herein described prior to addition to the remainder of the ingredients of the composition. Preferably, the oil soluble agent and the cooling agent are added last.

By oil soluble is meant any material that is immiscible with water.

The compounds disclosed above in all embodiments of the invention can be made according to a general process disclosed in WO 2004/026840 in particular by the process described in the examples on pages 16-21.

### EXAMPLES

The following toothpaste is made by emulsifying the cooling agent in the flavour component before adding to the remainder of the composition.

| Ingredient | % w/w |
|---|---|
| WATER DEM. CL. | 27.51 |
| NEOSORB 70/70 | 45.00 |
| SODIUM FLUORIDE | 0.32 |
| SODIUM SACCARIN | 0.17 |
| PEG 32 | 5.00 |
| MONOSODIUM PHOSPHATE | 0.10 |
| TITANIUM DIOXIDE | 1.00 |
| CMC 9M | 0.90 |
| TIXOSIL 43 | 7.50 |
| SORBOSIL AC 77 | 10.00 |
| SLS | 1.50 |
| AG35-CL | 0.05 |
| Flavour | 0.95 |
| | 100.00 |

The following mouthwash is made by emulsifying the cooling agent in the flavour component before adding to the remainder of the composition.

| Ingredient | % w/w |
|---|---|
| water | 92.12% |
| ethanol | 6.28% |
| pluronic F127 | 0.26% |
| Sodium lauryl sulphate | 1.07% |
| flavour | 0.21% |
| AG35Cl | 0.05% |

1. An oral care composition, comprising: a therapeutically effective amount of a cooling compound having the structure of Formula [I]: 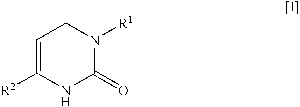
or a salt thereof to produce a cooling sensation, wherein R¹ and R² are independently selected from hydrogen or halogen atoms; hydroxy, cyano, nitro, mercapto, carbonyl, sulfone and carboxy groups; or optionally substituted alkyl, alkenyl, alkoxy, alkylthio, aryl, aryloxy, arylthio, amino, siloxy, ester and heterocyclic groups, characterised by an oil soluble agent for suspending, solubilising or emulsifying the cooling compound.

2. An oral care composition according to claim 1 wherein the oil soluble agent is a flavour.

3. An oral care composition according to claim 1 wherein the oil soluble agent comprises one or more flavoring components selected from oils and extracts of basil, camphor, caraway, cardamon, coriander, geranium, ginger, laurel, lavender, mace, menthol, nutmeg, pepper, rose, rosemary, thyme, ylang ylang, jasmin, vanilla, hyssop, lavandin, orris, carrot seed, davana, elemi and osmanthus; borneol and its derivatives; heliotropin, α-ionone, β-ionone, γ-ionone, δ-ionone and derivatives thereof; lactones and thymol, vanillin, ethyl vanillin, maltol and ethyl maltol.

4. An oral care composition according to claim 1 wherein the oil soluble agent is present at from 0.001 to 2% by weight of the composition.

5. An oral care composition according to claim 1 wherein the cooling is present in the composition at from 0.001 to 0.1% by weight of the composition.

6. An oral care composition according to claim 1 comprising a surfactant.

7. An oral care composition according to claim 1 comprising from 0.75 to 1.5% by weight surfactant.

8. An oral care composition according to claim 1 wherein the cooling compound is of the structure represented by Formula II: 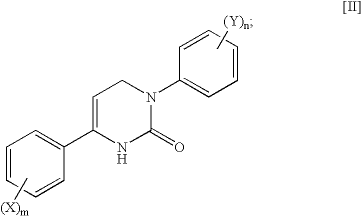
wherein the or each X and Y is independently a halogen atom or an alkyl, alkenyl, haloalkyl, alkoxy, hydroxy, thiol, carboxy, nitro, sulphonamide, sulphonato, sulphonyl, alkoxycarbonyl, carbonyl or amino group, and m and n are independently 0, 1, 2 or 3.

9. An oral care composition according to claim 1 wherein the cooling compound is of the structure represented by Formula III: 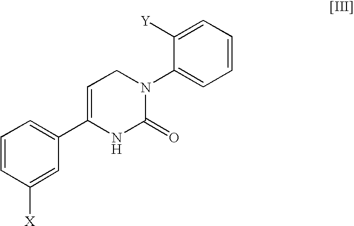
wherein X and Y are independently selected from a halogen atom or an alkyl, alkenyl, haloalkyl, alkoxy, hydroxy, thiol, carboxy, nitro or amino group.

10. An oral care composition according to claim 1 wherein the cooling compound is of formula [IV]: 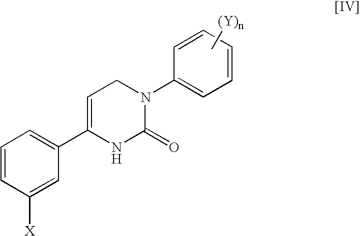
or a salt thereof, wherein X is a hydrogen or halogen atom, or a hydroxy, nitro, alkyl or alkoxy group; Y is hydrogen, hydroxy, haloalkyl, nitro or alkoxy; and n is 0, 1, 2 or 3.

11. An oral care composition according to claim 1 wherein the cooling compound particularly preferred compounds are those according to the general formula [V]: 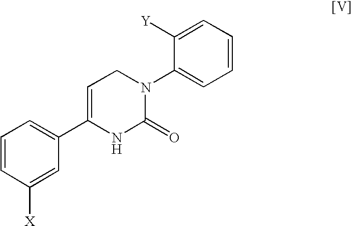
with Y being as defined above. It is preferred that Y is a hydroxy group or a methoxy group. X is preferably a hydrogen or halogen atom, or a hydroxy, C₁₋₆ alkyl or C₁₋₆ alkoxy group, providing that where Y is hydroxy, X is alkyl or hydroxy.

12. An oral care composition according to claim 1 wherein the cooling compound is 1-[2-hydroxyphenyl]-4-[3-nitrophenyl]-1,2,3,6-tetrahydropyrimidine-2-one (AG35) or an analog thereof.

13. An oral care composition according to claim 1 wherein the cooling compound is 1-(2′-hydroxyphenyl)-4-(3″-chlorophenyl)-1,2,3,6-tetrahydropyrimidine-2-one (AG35Cl).
